(19) United States
(12) Patent Application Publication
(10) Pub. No.: US 2024/0335199 A1
(43) Pub. Date: Oct. 10, 2024

(54) VASCULAR PLUG

(71) Applicant: ARIZONA BOARD OF REGENTS ON BEHALF OF THE UNIVERSITY OF ARIZONA, Tucson, AZ (US)

(72) Inventors: Craig C. Weinkauf, Tucson, AZ (US); Scott DeRigne, Tucson, AZ (US)

(21) Appl. No.: 18/294,074

(22) PCT Filed: Jun. 30, 2022
(86) PCT No.: PCT/US22/35850

§ 371 Date: Jan. 31, 2024

Related U.S. Application Data
(60) Provisional application No. 63/235,603, filed on Aug. 20, 2021.

(51) Int. Cl. A61B 17/12

(52) U.S. Cl. CPC A61B 17/1204; A61B 17/12109; A61B 17/12159

(57) ABSTRACT

Blood loss during surgery is inevitable. Currently, clamps may be used to try and stop the bleeding of blood vessels. However, some blood vessels cannot be adequately clamped and therefore are left to leak blood which is then sponged off. The present invention features devices and methods for occluding vascular vessels during surgery to help prevent blood loss. Specifically, the present invention features a device (i.e., a plug) that, when inserted into the vessel lumen of the vascular vessel, prevents blood flow by exerting a radial force on the vessel. The small and versatile plugs described herein help reduce blood loss during surgery and can work on all sizes of blood vessels.

### CROSS-REFERENCES TO RELATED APPLICATIONS

This application claims benefit of U.S. Provisional Application No. 63/235,603 filed Aug. 20, 2021, the specification of which is incorporated herein in their entirety by reference.

### FIELD OF THE INVENTION

The present invention features a device to occlude vascular vessels.

### BACKGROUND OF THE INVENTION

The World Health Organization estimates that 234.2 million surgical procedures are performed every year worldwide, with around 50 million of those procedures happening within the United States during recorded years. One of the main problems that are faced during surgery is blood loss of the patient. When a patient loses too much blood during surgery, it can result in permanent organ damage or even organ failure. Therefore, it is important to prevent blood loss during surgical procedures.

Current techniques to stop blood loss during surgeries are to use clamps on the bleeding site; however, there are some sites that are unable to be easily clasped. Bleeding can also be stopped by ligation and sutures of the bleeding vessels, which takes around five minutes for each vessel. The present invention features devices and methods to assist in the ligation process by plugging the vessels to allow work on one ligation at a time. The plugs described herein will help greatly reduce blood loss in surgery patients and are also small, versatile, and cost-effective solutions to this issue.

### BRIEF SUMMARY OF THE INVENTION

It is an objective of the present invention to provide devices and methods that allow for temporary occlusion of vascular vessels to quickly reduce blood loss, as specified in the independent claims. Embodiments of the invention are given in the dependent claims. Embodiments of the present invention can be freely combined with each other if they are not mutually exclusive.

Once a vessel is transected or opened, hemostasis needs to be obtained quickly yet sometimes the vessel can be ligated permanently and sometimes it needs to be repaired. The device described herein allows rapid hemostasis (i.e., about 5 seconds or less), which is much faster than suturing. Additionally, the device described herein removed, if needed, so the vessel can be repaired once hemostasis is obtained. The ability to remove the device is relevant in many aspects of surgery but particularly relevant in aortic surgery when needing to obtain rapid and sometimes reversible hemostasis with bleeding lumbar and segmental spinal arteries. Furthermore, there are instances when 6-12 small bleeding arteries need to be controlled in a very rapid fashion. Thus, devices described herein allow for quick and efficient occlusion of variously sized vascular vessels during surgery.

The present invention features a device for occluding a vascular vessel having a vessel lumen. The device may comprise a stem having a first end and a second end. The device may further comprise a plurality of discs disposed near or at the second end of the stem. The plurality of discs may be spaced longitudinally along the stem. In some embodiments, each disc comprises a top side and a bottom side. The top side is positioned towards the first end of the stem, and the bottom side is positioned towards the second end of the stem. In some embodiments, each disc has a radius and the radius of each disc remains the same or decreases toward the second end of the stem, such that the plurality of discs form a cone shape with the point of the cone at the second end of the stem. In some embodiments, the device is configured to prevent blood flow when inserted into the vessel lumen of the vascular vessel.

One of the unique and inventive technical features of the present invention is the design of the vascular plug device. For example, the device comprises a plurality of discs spaced longitudinally along a stem. Without wishing to limit the invention to any theory or mechanism, it is believed that the technical feature of the present invention advantageously provides for a temporary and cost-effective device for quick and efficient occlusion of variously sized vascular vessels during surgery. Additionally, the technical feature of the present invention advantageously maintains homeostasis, however, the vascular plug device is also soft enough to not cause significant arterial wall damage and can be removed as needed. Lastly the vascular plug device described herein can work with vessels that range from about 1 mm-5 mm in diameter. None of the presently known prior references or work has the unique inventive technical feature of the present invention.

Additionally, the prior arts such as those shown in FIG. 4 do not have particular features of the devices described herein. For example, U.S. Pat. No. 9,345,461B2 of Chin fails to teach of a plurality of discs spaced longitudinally along a stem and contrarily teaches a “mushroom-shaped sealing element.” Furthermore, neither U.S. Pat. No. 9,101,731B2 of Zhu nor U.S. Pat. No. 8,986,338B2 of Obermiller teaches of a plurality of discs spaced longitudinally along a stem. For instance, Zhu teaches a singular raised portion at the distal end of the device and requires the use of a sponge and/or a multilayer patch to inhibit bleeding. Lastly, WO1994006460A1 of Conston teaches a bioresorbable plug made from collagen but fails to teach of a stem or a plurality of discs. None of the prior arts teach a plug comprising a plurality of discs spaced longitudinally along a stem to be used to occlude blood vessels that have been cut during surgery.

Furthermore, the prior references teach away from the present invention. For example, during surgery procedures, bleeding is currently stopped by ligation and suture of the vessel, however, this can take up to 5 minutes. Additionally, some blood vessels are too small to clamp, and therefore the blood vessels are just left to leak blood which is then sponged off. This can ultimately impair a surgeon's ability to see during surgery. Moreover, other methods to occlude vascular vessels require inflation of the device to properly block the vascular vessel.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWINGS

The features and advantages of the present invention will become apparent from a consideration of the following detailed description presented in connection with the accompanying drawings in which:

FIG. 1 shows a non-limiting example of devices as described herein.

FIG. 2 shows an embodiment of a device as described herein with non-limiting dimensions.

FIG. 3 is a close-up of the device having a stem and a plurality of discs.

FIGS. 4A and 4B show a non-limiting example of a device as described herein (FIG. 4A) preventing blood flow from a vascular vessel once inserted into the vessel lumen (FIG. 4B).

FIG. 5 shows examples of devices described in the prior arts.

### DETAILED DESCRIPTION OF THE INVENTION

Following is a list of elements corresponding to a particular element referred to herein:
100 Vascular Plug Device
110 Stem
111 First end
112 Second end
120 Disc
121 Top side
122 Bottom side
125 Outer Edge

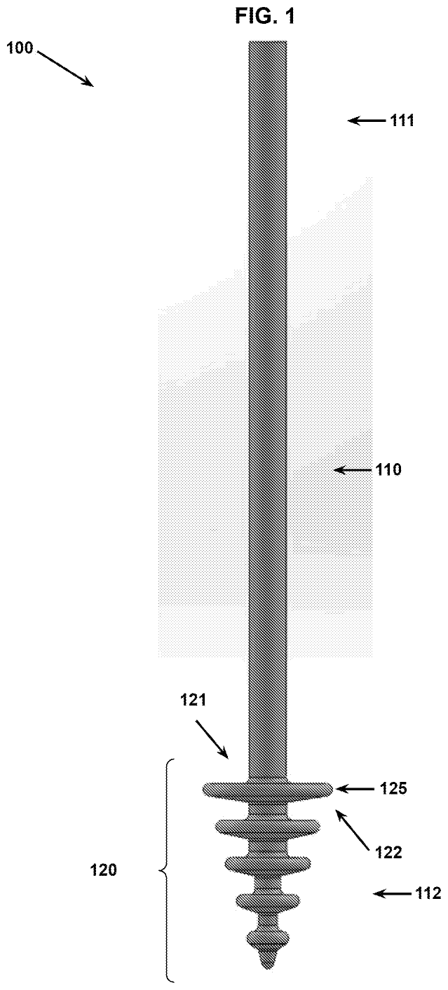

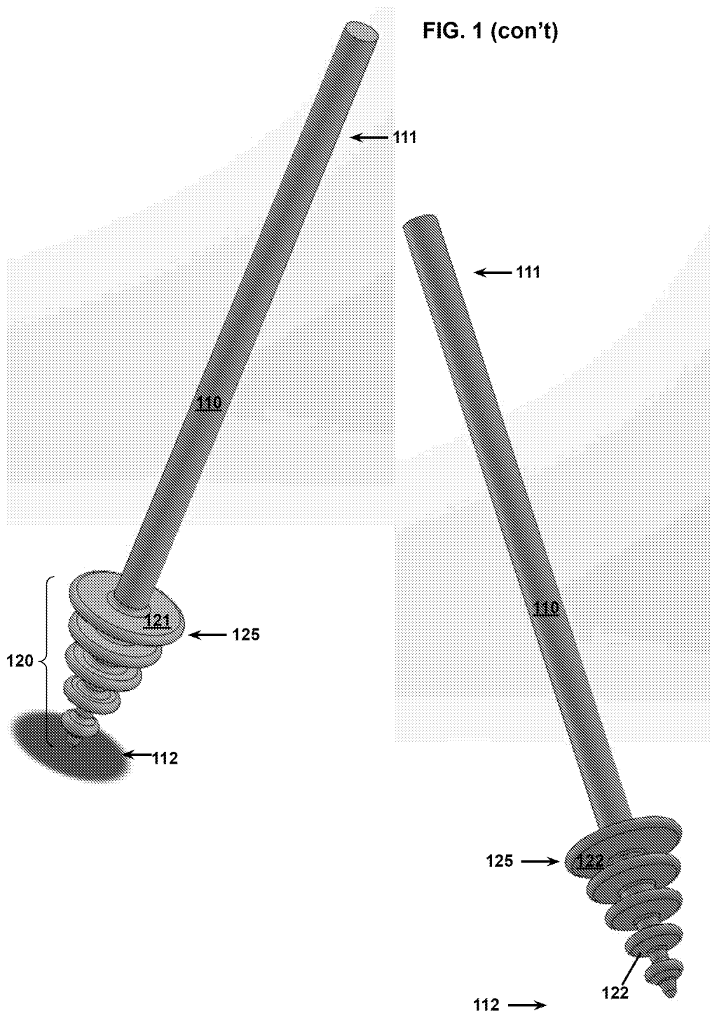

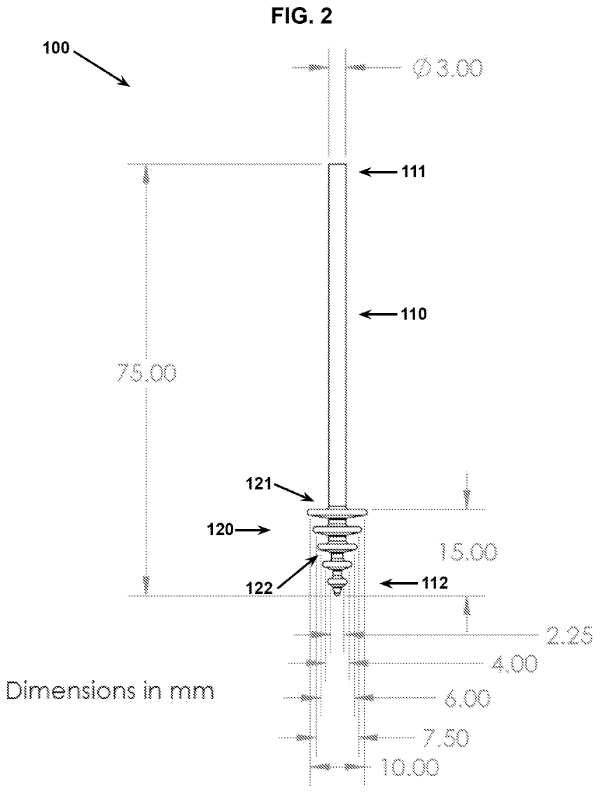

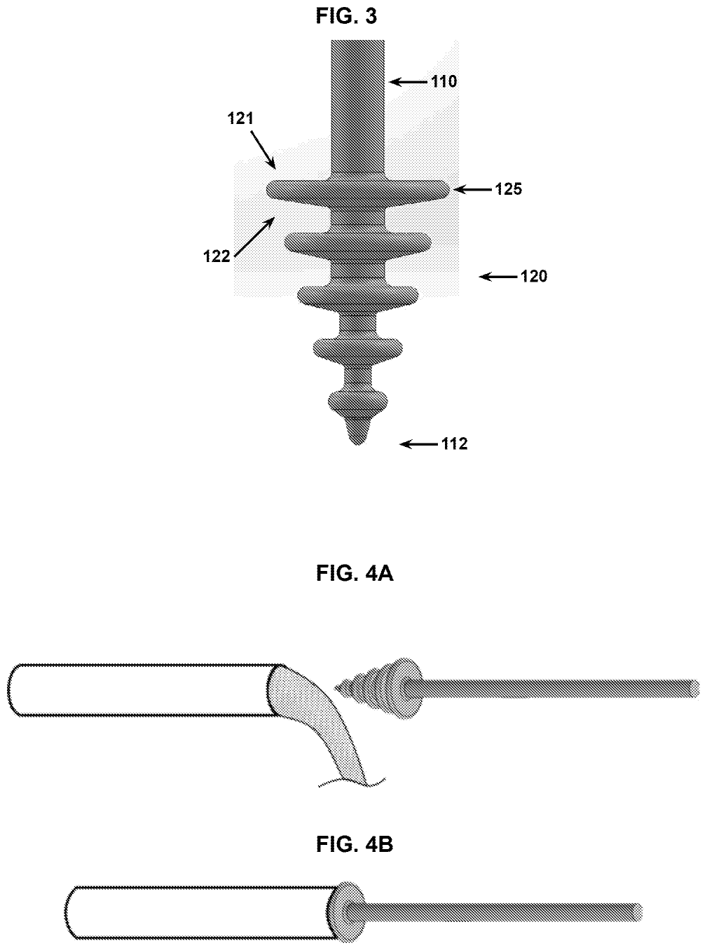

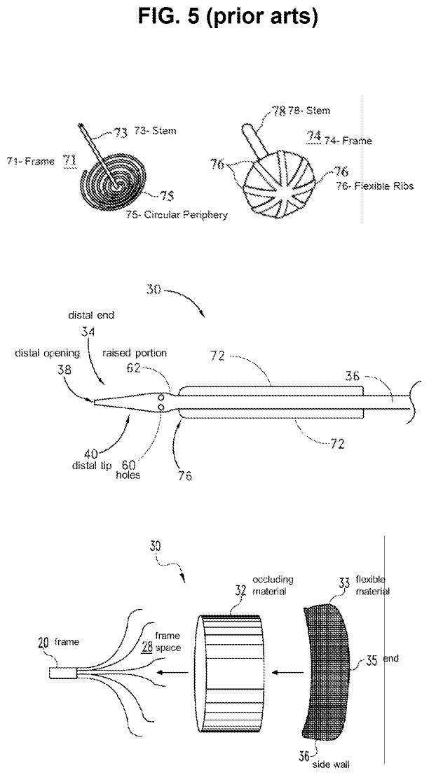

Referring now to FIG. 1, FIG. 2, FIG. 3, FIGS. 4A and 4B and FIG. 5, the present invention features devices and methods to occlude vascular vessels.

The present invention features a device (100) for occluding a vascular vessel having a vessel lumen. The device (100) may comprise a stem (110) having a first end (111) and a second end (112). The device (100) may further comprise a plurality of discs (120) disposed near and/or at the second end (112) of the stem (110). The plurality of discs (120) may be spaced longitudinally along the stem (110). In some embodiments, each disc (120) comprises a top side (121) and a bottom side (122). The top side (121) is positioned towards the first end (111) of the stem (110), and the bottom side (122) is positioned towards the second end (112) of the stem (110). In some embodiments, each disc (120) has a diameter, and the diameter of each disc (120) remains the same or decreases toward the second end (112) of the stem (110), such that the plurality of discs (120) form a cone shape with the point of the cone at the second end (112) of the stem (110). In some embodiments, the device (100) is configured to prevent blood flow when inserted into the vessel lumen of the vascular vessel.

In some embodiments, the vascular vessel is an artery. In other embodiments, the vascular vessel is a vein. In some embodiments, the vascular vessel comprises a dimeter ranging from about 1.0 mm to 5.0 mm, or about 1.0 mm to 4.5 mm, or about 1.0 mm to 4.0 mm, or about 1.0 mm to 3.5 mm, or about 1.0 mm to 3.0 mm, or about 1.0 mm to 2.5 mm, or about 1.0 mm to 2.0 mm, or about 1.0 mm to 1.5 mm, or about 1.5 mm to 5.0 mm, or about 1.5 mm to 4.5 mm, or about 1.5 mm to 4.0 mm, or about 1.5 mm to 3.5 mm, or about 1.5 mm to 3.0 mm, or about 1.5 mm to 2.5 mm, or about 1.5 mm to 2.0 mm, or about 2.0 mm to 5.0 mm, or about 2.0 mm to 4.5 mm, or about 2.0 mm to 4.0 mm, or about 2.0 mm to 3.5 mm, or about 2.0 mm to 3.0 mm, or about 2.0 mm to 2.5 mm, or about 2.5 mm to 5.0 mm, or about 2.5 mm to 4.5 mm, or about 2.5 mm to 4.0 mm, or about 2.5 mm to 3.5 mm, or about 2.5 mm to 3.0 mm, or about 3.0 mm to 5.0 mm, or about 3.0 mm to 4.5 mm, or about 3.0 mm to 4.0 mm, or about 3.0 mm to 3.5 mm, or about 3.5 mm to 5.0 mm, or about 3.5 mm to 4.5 mm, or about 3.5 mm to 4.0 mm, or about 4.0 mm to 5.0 mm, or about 4.0 mm to 4.5 mm, or about 4.5 mm to 5.0 mm.

In other embodiments, the vascular vessel comprises a diameter ranging from about 5.0 mm to 15 mm, or about 5.0 mm to 12 mm, or about 5.0 mm to 10 mm, or about 5.0 mm to 9.0 mm, or about 5.0 mm to 8.0 mm, or about 5.0 mm to 7.0 mm, or about 5.0 mm to 6.0 mm, or about 6.0 mm to 15 mm, or about 6.0 mm to 12 mm, or about 6.0 mm to 10 mm, or about 6.0 mm to 9.0 mm, or about 6.0 mm to 8.0 mm, or about 6.0 mm to 7.0 mm, or about 7.0 mm to 15 mm, or about 7.0 mm to 12 mm, or about 7.0 mm to 10 mm, or about 7.0 mm to 9.0 mm, or about 7.0 mm to 8.0 mm, or about 8.0 mm to 15 mm, or about 8.0 mm to 12 mm, or about 8.0 mm to 10 mm, or about 8.0 mm to 9.0 mm, or about 9.0 mm to 15 mm, or about 9.0 mm to 12 mm, or about 9.0 mm to 10 mm, or about 10 mm to 15 mm, or about 10 mm to 12 mm, or about 12 mm to 15 mm.

In other embodiments, the vascular vessel comprises a diameter of about 1.0 mm, or about 1.5 mm, or about 2.0 mm, or about 2.5 mm, or about 3.0 mm, or about 3.5 mm, or about 4.0 mm, or about 4.5 mm, or about 5.0 mm, or about 6.0 mm, or about 7.0 mm, or about 8 mm, or about 9 mm, or about 10 mm, or about 12 mm, or about 15 mm.

In some embodiments, the stem (110) is cylindrical. In other embodiments, the stem (110) is rectangular. In some embodiments, the stem (110) is a bulb shape, wherein the widest part of the bulb is at the first end (111) of the stem (110). In preferred embodiments, the stem (110) is solid. Without wishing to limit the present invention to any theories or mechanisms it is believed that a solid stem (110) will allow for the stem (110) to be removed from a mold without damaging said stem (110). In other embodiments, the stem is hollow.

In preferred embodiments, the stem (110; e.g., a cylindrical stem) comprises a radius of about 3.0 mm. In some embodiments, the stem (110; e.g., a cylindrical stem) comprises a radius of about 1.5 mm, or about 2.0 mm, or about 2.5 mm. In some embodiments, the stem (110; e.g., a cylindrical stem) comprises a radius of about 3.5 mm, or about 4.0 mm, or about 4.5 mm, or about 5.0 mm, or about 5.5 mm. In some embodiments, the stem (110; e.g., a cylindrical stem) comprises a radius of 1.5 mm, 2.0 mm, 2.5 mm, 3.0 mm, 3.5 mm, 4.0 mm, 4.5 mm, 5.0 mm, or 5.5 mm.

In some embodiments, the radius of the stem (110; e.g., a cylindrical stem) is about 1.5 mm to 5.5 mm, or about 1.5 mm to 5.0 mm, or about 1.5 mm to 4.5 mm, or about 1.5 mm to 4.0 mm, or about 1.5 mm to 3.5 mm, or about 1.5 mm to 3.0 mm, or about 1.5 mm to 2.5 mm, or about 1.5 mm to 2.0 mm, or about 2.0 mm to 5.5 mm, or about 2.0 mm to 5.0 mm, or about 2.0 mm to 4.5 mm, or about 2.0 mm to 4.0 mm, or about 2.0 mm to 3.5 mm, or about 2.0 mm to 3.0 mm, or about 2.0 mm to 2.5 mm, or about 2.5 mm to 5.5 mm, or about 2.5 mm to 5.0 mm, or about 2.5 mm to 4.5 mm, or about 2.5 mm to 4.0 mm, or about 2.5 mm to 3.5 mm, or about 2.5 mm to 3.0 mm, or about 3.0 mm to 5.5 mm, or about 3.0 mm to 5.0 mm, or about 3.0 mm to 4.5 mm, or about 3.0 mm to 4.0 mm, or about 3.0 mm to 3.5 mm, or about 3.5 mm to 5.5 mm, or about 3.5 mm to 5.0 mm, or about 3.5 mm to 4.5 mm, or about 3.5 mm to 4.0 mm, or about 4.0 mm to 5.5 mm, or about 4.0 mm to 5.0 mm, or about 4.0 mm to 4.5 mm, or about 4.5 mm to 5.5 mm, or about 4.5 mm to 5.0 mm, or about 5.0 mm to 5.5 mm.

In preferred embodiments, the stem (110; e.g., a rectangular stem) has a width of about 3.0 mm. In some embodiments, the stem (110; e.g., a rectangular stem) has a width of about 1.5 mm, or about 2.0 mm, or about 2.5 mm. In some embodiments, the stem (110; e.g., a rectangular stem) has a width of about 3.5 mm, or about 4.0 mm, or about 4.5 mm, or about 5.0 mm, or about 5.5 mm. In some embodiments, the stem (110) has a width of 1.5 mm, 2.0 mm, 2.5 mm, 3.0 mm, 3.5 mm, 4.0 mm, 4.5 mm, 5.0 mm, or 5.5 mm.

In some embodiments, the width of the stem (110; e.g., a rectangular stem) is about 1.5 mm to 5.5 mm, or about 1.5 mm to 5.0 mm, or about 1.5 mm to 4.5 mm, or about 1.5 mm to 4.0 mm, or about 1.5 mm to 3.5 mm, or about 1.5 mm to 3.0 mm, or about 1.5 mm to 2.5 mm, or about 1.5 mm to 2.0 mm, or about 2.0 mm to 5.5 mm, or about 2.0 mm to 5.0 mm, or about 2.0 mm to 4.5 mm, or about 2.0 mm to 4.0 mm, or about 2.0 mm to 3.5 mm, or about 2.0 mm to 3.0 mm, or about 2.0 mm to 2.5 mm, or about 2.5 mm to 5.5 mm, or about 2.5 mm to 5.0 mm, or about 2.5 mm to 4.5 mm, or about 2.5 mm to 4.0 mm, or about 2.5 mm to 3.5 mm, or about 2.5 mm to 3.0 mm, or about 3.0 mm to 5.5 mm, or about 3.0 mm to 5.0 mm, or about 3.0 mm to 4.5 mm, or about 3.0 mm to 4.0 mm, or about 3.0 mm to 3.5 mm, or about 3.5 mm to 5.5 mm, or about 3.5 mm to 5.0 mm, or about 3.5 mm to 4.5 mm, or about 3.5 mm to 4.0 mm, or about 4.0 mm to 5.5 mm, or about 4.0 mm to 5.0 mm, or about 4.0 mm to 4.5 mm, or about 4.5 mm to 5.5 mm, or about 4.5 mm to 5.0 mm, or about 5.0 mm to 5.5 mm.

In preferred embodiments, the stem (110) is about 60 mm in length. In some embodiments, the stem (110) is about 30 mm in length, or about 35 mm in length. In some embodiments, the stem (110) is about 40 mm in length, or about 45 mm in length, or about 50 mm in length, or about 55 mm in length. In some embodiments, the stem (110) is about 65 mm in length, or about 70 mm in length, or about 75 mm in length, or about 80 mm in length, or about 85 mm in length. In some embodiments, the stem (110) is 30 mm, 35 mm, 40 mm, 45 mm, 50 mm, 55 mm, 60 mm, 65 mm, 70 mm, 75 mm, 80 mm, or 85 mm in length.

In some embodiments, the length of the stem (110) is about 30 mm to 85 mm, or about 30 mm to 80 mm, or about 30 mm to 75 mm, or about 30 mm to 70 mm, or about 30 mm to 65 mm, or about 30 mm to 60 mm, or about 30 mm to 55 mm, or about 30 mm to 50 mm, or about 30 mm to 45 mm, or about 30 mm to 40 mm, or about 30 mm to 35 mm, or about 35 mm to 85 mm, or about 35 mm to 80 mm, or about 35 mm to 75 mm, or about 35 mm to 70 mm, or about 35 mm to 65 mm, or about 35 mm to 60 mm, or about 35 mm to 55 mm, or about 35 mm to 50 mm, or about 35 mm to 45 mm, or about 35 mm to 40 mm, or about 40 mm to 85 mm, or about 40 mm to 80 mm, or about 40 mm to 75 mm, or about 40 mm to 70 mm, or about 40 mm to 65 mm, or about 40 mm to 60 mm, or about 40 mm to 55 mm, or about 40 mm to 50 mm, or about 40 mm to 45 mm, or about 45 mm to 85 mm, or about 45 mm to 80 mm, or about 45 mm to 75 mm, or about 45 mm to 70 mm, or about 45 mm to 65 mm, or about 45 mm to 60 mm, or about 45 mm to 55 mm, or about 45 mm to 50 mm, or about 50 mm to 85 mm, or about 50 mm to 80 mm, or about 50 mm to 75 mm, or about 50 mm to 70 mm, or about 50 mm to 65 mm, or about 50 mm to 60 mm, or about 50 mm to 55 mm, or about 55 mm to 85 mm, or about 55 mm to 80 mm, or about 55 mm to 75 mm, or about 55 mm to 70 mm, or about 55 mm to 65 mm, or about 55 mm to 60 mm, or about 60 mm to 85 mm, or about 60 mm to 80 mm, or about 60 mm to 75 mm, or about 60 mm to 70 mm, or about 60 mm to 65 mm, or about 65 mm to 85 mm, or about 65 mm to 80 mm, or about 65 mm to 75 mm, or about 65 mm to 70 mm, or about 70 mm to 85 mm, or about 70 mm to 80 mm, or about 70 mm to 75 mm, or about 75 mm to 85 mm, or about 75 mm to 80 mm, or about 80 mm to 85 mm.

In some embodiments, the device comprises 2 discs (120). In some embodiments, the device comprises 3 discs (120). In some embodiments, the device comprises 4 discs (120). In some embodiments, the device comprises 5 discs (120). In some embodiments, the device comprises 6 discs (120). In some embodiments, the device comprises 7 discs (120). In some embodiments, the device comprises 8 discs (120). In some embodiments, the device (100) comprises discs (120) that are the same size. In other embodiments, the device (100) comprises discs (120) that are all different sizes. In further embodiments, the device (100) comprises discs (120) that are a combination of the same sized discs (120) and different sized discs (120).

In some embodiments, the plurality of discs (120) have a diameter ranging from about 0.5 mm to 15 mm, or about 0.5 mm to 10 mm, or about 0.5 mm to 5 mm, or about 0.5 mm to 1 mm. In other embodiments, the plurality of discs (120) have a diameter ranging from about 1 mm to 15 mm, or about 1 mm to 10 mm, or about 1 mm to 5 mm.

In some embodiments, the diameter of the plurality of discs (120) ranges from about 0.5 mm to 15 mm, or about 0.5 mm to 12 mm, or about 0.5 mm to 10 mm, or about 0.5 mm to 8 mm, or about 0.5 mm to 7.5 mm, or about 0.5 mm to 7.0 mm, or about 0.5 mm to 6.0 mm, or about 0.5 mm to 5.0 mm, or about 0.5 mm to 4.0 mm, or about 0.5 mm to 3.0 mm or about 0.5 mm to 2.0 mm, or about 0.5 mm to 1.0 mm, or about 1.0 mm to 15 mm, or about 1.0 mm to 12 mm, or about 1.0 mm to 10 mm, or about 1.0 mm to 8 mm, or about 1.0 mm to 7.5 mm, or about 1.0 mm to 7.0 mm, or about 1.0 mm to 6.0 mm, or about 1.0 mm to 5.0 mm, or about 1.0 mm to 4.0 mm, or about 1.0 mm to 3.0 mm or about 1.0 mm to 2.0 mm, or about 2.0 mm to 15 mm, or about 2.0 mm to 12 mm, or about 2.0 mm to 10 mm, or about 2.0 mm to 8 mm, or about 2.0 mm to 7.5 mm, or about 2.0 mm to 7.0 mm, or about 2.0 mm to 6.0 mm, or about 2.0 mm to 5.0 mm, or about 2.0 mm to 4.0 mm, or about 1.0 mm to 3.0 mm or about 3.0 mm to 15 mm, or about 3.0 mm to 12 mm, or about 3.0 mm to 10 mm, or about 3.0 mm to 8 mm, or about 3.0 mm to 7.5 mm, or about 3.0 mm to 7.0 mm, or about 3.0 mm to 6.0 mm, or about 3.0 mm to 5.0 mm, or about 3.0 mm to 4.0 mm, or about 4.0 mm to 15 mm, or about 4.0 mm to 12 mm, or about 4.0 mm to 10 mm, or about 4.0 mm to 8 mm, or about 4.0 mm to 7.5 mm, or about 4.0 mm to 7.0 mm, or about 4.0 mm to 6.0 mm, or about 4.0 mm to 5.0 mm, or about 5.0 mm to 15 mm, or about 5.0 mm to 12 mm, or about 5.0 mm to 10 mm, or about 5.0 mm to 8 mm, or about 5.0 mm to 7.5 mm, or about 5.0 mm to 7.0 mm, or about 5.0 mm to 6.0 mm, or about 6.0 mm to 15 mm, or about 6.0 mm to 12 mm, or about 6.0 mm to 10 mm, or about 6.0 mm to 8 mm, or about 6.0 mm to 7.5 mm, or about 6.0 mm to 7.0 mm, or about 7.0 mm to 15 mm, or about 7.0 mm to 12 mm, or about 7.0 mm to 10 mm, or about 7.0 mm to 8 mm, or about 7.0 mm to 7.5 mm, or about 7.5 mm to 15 mm, or about 7.5 mm to 12 mm, or about 7.5 mm to 10 mm, or about 7.5 mm to 8 mm, or about 8.0 mm to 15 mm, or about 8.0 mm to 12 mm, or about 8.0 mm to 10 mm, or about 10 mm to 15 mm, or about 10 mm to 12 mm, or about 12 mm to 15 mm.

In certain embodiments, the device (100) comprises 6 discs (120) attached to the second end (112) of the stem (110). In some embodiments, the diameter of the discs (120) starting at the tip of the second end (112) of the stem (100) are 1 mm, 2.25 mm, 4.00 mm, 6.00 mm, 7.5 mm, and 10 mm.

In some embodiments, the top side (121) of the plurality of discs (120) is flat. In other embodiments, the bottom side (122) of the plurality of discs (120) is convexed. Without wishing to limit the present invention to any theory or mechanism it is believed that devices (100) comprising a plurality of discs (120) in which the top side (121) is flat and the bottom side (122) is convexed allows for some flexure when inserting the device (100) in a vessel and provides resistance to the force of the blood pressure exerted on the device (100).

In some embodiments, an outer edge (125) of each disc (120) is convex and smooth.

In some embodiments, each disc (120) has an outer edge (125). In some embodiments, the outer edge (125) of the disc (120) is about 0.5 mm thick, or about 0.75 mm, or about 1.0 mm thick, or about 1.25 mm thick, or about 1.5 mm thick, or about 1.75 mm thick, or about 2.0 mm thick, or about 2.5 mm thick, or about 3.0 mm thick, or about 3.5 mm thick, or about 4.0 mm thick. In some embodiments, the thickness of the outer edge (125) of a disc (120) ranges from about 0.50 mm to 4.0 mm, or about 0.50 mm to 3.5 mm, about 0.50 mm to 3.0 mm, or about 0.50 mm to 2.5 mm, or about 0.50 mm to 2.0 mm, or about 0.50 mm to 1.75 mm, or about 0.50 mm to 1.5 mm, or about 0.50 mm to 1.25 mm, or about 0.50 mm to 1.0 mm, or about 0.50 mm to 0.75 mm, or about 0.75 mm to 4.0 mm, or about 0.75 mm to 3.5 mm, or about 0.75 mm to 3.0 mm, or about 0.75 mm to 2.5 mm, or about 0.75 mm to 2.0 mm, or about 0.75 mm to 1.75 mm, or about 0.75 mm to 1.5 mm, or about 0.75 mm to 1.25 mm, or about 0.75 mm to 1.0 mm, or about 1.0 mm to 4.0 mm, or about 1.0 mm to 3.5 mm, or about 1.0 mm to 3.0 mm, or about 1.0 mm to 2.5 mm, or about 1.0 mm to 2.0 mm, or about 1.0 mm to 1.75 mm, or about 1.0 mm to 1.5 mm, or about 1.0 mm to 1.25 mm, or about 1.25 mm to 4.0 mm, or about 1.25 mm to 3.5 mm, or about 1.25 mm to 3.0 mm, or about 1.25 mm to 2.5 mm, or about 1.25 mm to 2.0 mm, or about 1.25 mm to 1.75 mm, or about 1.25 mm to 1.5 mm, or about 1.5 mm to 4.0 mm, or about 1.5 mm to 3.5 mm, or about 1.5 mm to 3.0 mm, or about 1.5 mm to 2.5 mm, or about 1.5 mm to 2.0 mm, or about 1.5 mm to 1.75 mm, or about 1.75 mm to 4.0 mm, or about 1.75 to 3.5 mm, or about 1.75 mm to 3.0 mm, or about 1.75 mm to 2.5 mm, or about 1.75 mm to 2.0 mm, or about 2.0 mm to 4.0 mm, or about 2.0 mm to 3.5 mm, or about 2.0 mm to 3.0 mm, or about 2.0 mm to 2.5 mm, or about 2.5 mm to 4.0 mm, or about 2.5 mm to 3.5 mm, or about 2.5 mm to 3.0 mm, or about 3.0 mm to 4.0 mm, or about 3.0 mm to 3.5 mm, or about 3.5 mm to 4.0 mm. In some embodiments, the thicknesses of the outer edges (125) of the plurality of discs (120) are all the same. In other embodiments, the thicknesses of the outer edges (125) of the plurality of discs (120) are all different.

In some embodiments, the middle of the disc (120; where the disc (120) meets the stem) is about 1.0 mm thick, or about 1.25 mm thick, or about 1.5 mm thick, or about 1.75 mm thick, or about 2.0 mm thick, or about 2.5 mm thick, or about 3.0 mm thick, or about 3.5 mm thick, or about 4.0 mm thick, or 4.5 mm thick, or 5.0 mm thick. In some embodiments, the thickness of the disc (120) where said disc (120) meets the stem (i.e., the middle of the disc (120)) ranges from about 1.0 mm to 5.0 mm, or about 1.0 mm to 4.5 mm, 1.0 mm to 4.0 mm, or about 1.0 mm to 3.5 mm, or about 1.0 mm to 3.0 mm, or about 1.0 mm to 2.5 mm, or about 1.0 mm to 2.0 mm, or about 1.0 mm to 1.75 mm, or about 1.0 mm to 1.5 mm, or about 1.0 mm to 1.25 mm, or about 1.25 mm to 5.0 mm, or about 1.25 mm to 4.5 mm, 1.25 mm to 4.0 mm, or about 1.25 mm to 3.5 mm, or about 1.25 mm to 3.0 mm, or about 1.25 mm to 2.5 mm, or about 1.25 mm to 2.0 mm, or about 1.25 mm to 1.75 mm, or about 1.25 mm to 1.5 mm, or about 1.5 mm to 5.0 mm, or about 1.5 mm to 4.5 mm, 1.5 mm to 4.0 mm, or about 1.5 mm to 3.5 mm, or about 1.5 mm to 3.0 mm, or about 1.5 mm to 2.5 mm, or about 1.5 mm to 2.0 mm, or about 1.5 mm to 1.75 mm, or about 1.75 mm to 5.0 mm, or about 1.75 mm to 4.5 mm, 1.75 mm to 4.0 mm, or about 1.75 to 3.5 mm, or about 1.75 mm to 3.0 mm, or about 1.75 mm to 2.5 mm, or about 1.75 mm to 2.0 mm, or about 2.0 mm to 5.0 mm, or about 2.0 mm to 4.5 mm, 2.0 mm to 4.0 mm, or about 2.0 mm to 3.5 mm, or about 2.0 mm to 3.0 mm, or about 2.0 mm to 2.5 mm, or about 2.5 mm to 5.0 mm, or about 2.5 mm to 4.5 mm, 2.5 mm to 4.0 mm, or about 2.5 mm to 3.5 mm, or about 2.5 mm to 3.0 mm, or about 3.0 mm to 5.0 mm, or about 3.0 mm to 4.5 mm, 3.0 mm to 4.0 mm, or about 3.0 mm to 3.5 mm, or about 3.5 mm to 5.0 mm, or about 3.0 mm to 4.5 mm, 3.5 mm to 4.0 mm or about 4.0 mm to 5.0 mm, or about 4.0 mm to 4.5 mm, or about 4.5 mm to 5.0 mm.

In some embodiments, the middle of the disc (120; i.e., where the disc (120) meets the stem) is thicker than the outer edge (125) of the disc (120). Without wishing to limit the present invention to any theory or mechanism it is believed that the contour of the bottom side (122) of the disc (120) provides for more bending when inserting the device (100) into a vessel and the flat side of the top side (121) of the disc (120) resists the blood pressure exerted on the device (100).

In preferred embodiments, the plurality of discs (120) are 1.0 mm thick at the outer edge (125) and 1.5 mm thick where it meets the stem (110).

In some embodiments, the top side (121) of the plurality of discs (120) is more rigid than the bottom side (122) of the plurality of discs (120). In some embodiments, the plurality of discs (120) are more flexible when force is applied from the bottom side (122) towards the top side (121) of the discs (120).

In some embodiments, there are subtle differences in the contour of the top side (121) of the disc (120) and bottom side (122) of the disc (120) for each of the plurality of disc (120). The intent being to allow a certain amount of flexure when the device (100) is being inserted into the vessel but provide a resistance to the force exerted on the device (100) by the pressure from the blood.

In some embodiments, the plurality of discs (120) are stationary on the stem (110). In some embodiments, the plurality of discs (120) are separated by about 2 mm, or about 1.75 mm, or about 1.5 mm, or about 1.25 mm, or about 1.0 mm, or about 0.75 mm, or about 0.5 mm. In some embodiments, the plurality of discs (120) are separated by about 0.5 mm to 2 mm, or about 0.5 mm to 1.75 mm, or about 0.5 mm to 1.5 mm or about 0.5 mm to 1.25 mm, or about 0.5 mm to 1.0 mm or about 0.5 mm to 0.75 mm, or about 0.75 mm to 2 mm, or about 0.75 mm to 1.75 mm, or about 0.75 mm to 1.5 mm or about 0.75 mm to 1.25 mm, or about 0.75 mm to 1.0 mm, or about 1.0 mm to 2 mm, or about 1.0 mm to 1.75 mm, or about 1.0 mm to 1.5 mm or about 1.0 mm to 1.25 mm, or about 1.25 mm to 2 mm, or about 1.25 mm to 1.75 mm, or about 1.25 mm to 1.5 mm, or about 1.5 mm to 2 mm, or about 1.5 mm to 1.75 mm, or about 1.75 mm to 2 mm.

In some embodiments, the stem (110) and the plurality of discs (120) are made of the same material such that the device (100) can be fabricated as a monolithic part that does not require bonding. In some embodiments, the device (100) as described herein is a monolithic device. In other embodiments, the stem (110) and the plurality of discs (120) are made from different material.

In some embodiments, the device (100) as described herein is made of rubber. In other embodiments, the device (100) as described herein is made of medical grade silicon. In further embodiments, the device (100) as described herein is made of non-medical grade silicon. In accordance with the present invention, materials used in other medical applications that meet the requirements of the devices described herein may be used to make said devices (100). In some embodiments, the device (100) as described herein is made via injection moulding.

In some embodiments, the first end (111) of the stem (110) and a plurality of discs (120) attached to the second end (112) of the stem (110) have the same durometer. In other embodiments, the first end (111) of the stem (110) and a plurality of discs (120) attached to the second end (112) of the stem (110) have different durometers.

In some embodiments, the durometer of the device may be about 45 A Shore, or about 40 A Shore, or about 35 A Shore, or about 30 A Shore, or about 25 A Shore, or about 20 A Shore, or about 15 A Shore. In some embodiments, the durometer of the device ranges from 15 A Shore to 45 A Shore, or about 15 A Shore to 40 A Shore, or about 15 A Shore to 35 A Shore, or about 15 A Shore to 30 A Shore, or about 15 A Shore to 25 A Shore, or about 15 A Shore to 20 A Shore, or about 20 A Shore to 45 A Shore, or about 20 A Shore to 40 A Shore, or about 20 A Shore to 35 A Shore, or about 20 A Shore to 30 A Shore, or about 20 A Shore to 25 A Shore, or about 25 A Shore to 45 A Shore, or about 25 A Shore to 40 A Shore, or about 25 A Shore to 35 A Shore, or about 25 A Shore to 30 A Shore, or about 30 A Shore to 45 A Shore, or about 30 A Shore to 40 A Shore, or about 30 A Shore to 35 A Shore, or about 35 A Shore to 45 A Shore, or about 35 A Shore to 40 A Shore, or about 40 A Shore to 45 A Shore.

The devices (100) described herein will be flexible and compliant to a certain extent; however, the devices (100) will not appreciably compress with the forces expected to be exerted by the blood vessel.

In some embodiments, the present invention features a method of occluding a vascular vessel having a vessel lumen. In some embodiments, the method comprises inserting a device (100) as described herein into the lumen of a vessel. In some embodiments, the vessel has an open end into which at least one of the discs (120) is inserted, thereby plugging said end (FIGS. 3A and 3B).

In some embodiments, the force needed to insert the device (100) is less than the force needed to remove the device (100). In other embodiments, the device inserted into the vessel lumen exerts a radial force on the vessel to prevent blood from flowing out of the plugged vessel end. In some embodiments, the radial force exerted will be a function of which of the plurality of discs (120) of the device (100) is engaged in occluding the vessel and the size of the blood vessel.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

1. A device (100) for occluding a vascular vessel having a vessel lumen, the device comprising;
a. a stem (110) having a first end (111) and a second end (112); and
b. a plurality of discs (120) disposed near the second end (112) of the stem (110), wherein the plurality of discs (120) are spaced longitudinally along the stem (110), wherein each disc (120) comprises a top side (121) and a bottom side (122);
wherein the top side (121) is positioned towards the first end (111) of the stem (110), and the bottom side (122) is positioned towards the second end (112) of the stem (110);
wherein each disc (120) has a radius, wherein the radius of each disc (120) remains the same or decreases toward the second end (112) of the stem (110), such that the plurality of discs (120) form a cone shape with the point of the cone at the second end (112) of the stem (110);
wherein the device (100) is configured to prevent blood flow when inserted into the vessel lumen of the vascular vessel.

2. The device (100) of claim 1, wherein the stem (110) is cylindrical.

3. The device (100) of claim 1, wherein the stem (110) has a radius of about 1.5 mm to 3 mm.

4. The device (100) of claim 1, wherein the stem (110) is about 50 mm to 60 mm length.

5. The device (100) of claim 1, wherein the device (100) comprises 6 discs (120).

6. The device (100) of claim 1, wherein the device (100) comprises 5 discs (120).

7. The device (100) of claim 1, wherein the plurality of discs (120) have diameters ranging from 0.5 mm to 15 mm

8. The device (100) of claim 1, wherein the plurality of discs (120) have diameters ranging from 1 mm to 10 mm.

9. The device (100) of claim 1, wherein the top side (121) of the plurality of discs (120) is flat.

10. The device (100) of claim 1, wherein the bottom side (122) of the plurality of discs (120) is convexed.

11. The device (100) of claim 1, wherein an outer edge (125) of each disc (120) is convex and smooth.

12. The device (100) of claim 1, wherein the top side (121) of the plurality of discs (120) is more rigid than the bottom side (122) of the plurality of discs (120).

13. The device (100) of claim 1, wherein the plurality of discs (120) are more flexible when force is applied from the bottom side (122) towards the top side (121) of the discs (120).

14. The device (100) of claim 1, wherein the plurality of discs (120) are stationary on the stem (110).

15. The device (100) of claim 1, wherein the plurality of discs (120) are spaced about 1.0 mm to 1.5 mm apart.

16. The device (100) of claim 1, wherein the device is made from medical grade silicon.

17. The device (100) of claim 1, wherein the device is a monolithic device.

18. A method of occluding a vascular vessel having a vessel lumen, the method comprising inserting a device according to claim 1 into the lumen of a vessel, wherein the vessel has an open end into which at least one of the discs (120) is inserted therein, thereby plugging said end.

19. The method of claim 18, wherein the force needed to insert the device is less than the force needed to remove the device.

20. The method of claim 18, wherein the device inserted into the vessel lumen exerts a radial force on the vessel to prevent blood from flowing out of the plugged vessel end.
